(19) United States
(12) Patent Application Publication
(10) Pub. No.: US 2025/0216311 A1
(43) Pub. Date: Jul. 3, 2025

(54) CORE FLOODING FOR ELECTRICALLY IMPROVED OIL RECOVERY STUDY

(71) Applicant: Saudi Arabian Oil Company, Dhahran (SA)

(72) Inventors: Dong Kyu Cha, Dhahran (SA); Salah Hamad Al-Saleh, Dhahran (SA); Ghaida Aljuhani, Dammam (SA); Subhash Chandrabose Ayirala, Dhahran (SA); Abdulaziz S. Qasim, Dammam (SA); Ali Abdallah Al-Yousef, Dhahran (SA)

(21) Appl. No.: 18/402,118

(22) Filed: Jan. 2, 2024

(51) Int. Cl. G01N 15/08; G01N 1/40; G01N 1/44; G01N 33/24

(52) U.S. Cl. CPC G01N 15/0806; G01N 1/4077; G01N 1/44; G01N 15/0893; G01N 33/24; G01N 2001/4083

(57) ABSTRACT

A first electrical resistance of a core sample placed in a core sample holder is measured. The core sample is flushed with oil to displace gas and saturate the core sample with oil. A second electrical resistance of the core sample saturated with oil is measured. The core sample is heated and pressurized. A third electrical resistance of the core sample is measured. The core sample is flushed with an aqueous fluid to displace at least a portion of the oil from the core sample. A fourth electrical resistance of the core sample is measured. An amount of oil displaced from the core sample is measured.

### TECHNICAL FIELD

This disclosure relates to core flooding, and in particular, for electrically improved oil recovery studies.

### BACKGROUND

Hydrocarbon production from rock formations can include multiple stages of oil recovery. The first stage of hydrocarbon production, also referred to as primary recovery, involves utilizing natural reservoir energy to drive hydrocarbons from the reservoir, into the wellbore, and up to the surface. The primary recovery stage can reach its end when the available energy in the reservoir is depleted. During primary recovery, typically only around 10% of the initial hydrocarbons in the reservoir is recovered. The second stage of hydrocarbon production, also referred to as secondary recovery, involves injecting an external fluid such as water or gas into the reservoir through injection wells in order to maintain reservoir pressure and displace hydrocarbons toward the wellbore. Common secondary recovery methods include gas injection and waterflooding. The secondary recovery stage can reach its end when the injected fluid is produced in considerable amounts from the production wells. During secondary recovery, additional hydrocarbons can be recovered from the reservoir. The third stage of hydrocarbon production, also referred to as tertiary recovery or enhanced oil recovery, involves any recovery method that follows secondary recovery, such as waterflooding or pressure maintenance. During tertiary recovery, additional hydrocarbons can be recovered from the reservoir.

### SUMMARY

This disclosure describes technologies relating to core flooding experiments that implements electrical current for studying fluid flow through porous media. Certain aspects of the subject matter described can be implemented as a method. The method includes measuring a first electrical resistance of a core sample placed in a core sample holder. The core sample is obtained from a subterranean formation containing hydrocarbons. The core sample is in a dry state. A pore volume of the core sample is at least partially filled with gas. The method includes flushing the core sample with oil to displace gas from the core sample and saturate the core sample with the oil. The method includes, after saturating the core sample with the oil, measuring a second electrical resistance of the core sample saturated with the oil. The method includes heating the core sample to a downhole reservoir temperature. The method includes pressurizing the oil in the core sample to a downhole reservoir pressure. The method includes, after pressurizing the oil, measuring a third electrical resistance of the core sample. The method includes flushing the core sample with an aqueous fluid to displace at least a portion of the oil from the core sample. The method includes, while flushing the core sample with the aqueous fluid, measuring a fourth electrical resistance of the core sample and measuring an amount of the oil displaced from the core sample.

In some implementations, the method includes determining a change in pore throat of the core sample, permeability of the core sample, or both based on the measured first, second, third, and fourth electrical resistances of the core sample.

In some implementations, the method includes, prior to measuring the first electrical resistance of the core sample: wrapping the core sample in an insulating blanket, surrounding the wrapped core sample with a polymer sleeve, and placing the wrapped core sample surrounded by the polymer sleeve in the core sample holder.

In some implementations, the method includes, prior to measuring the first electrical resistance of the core sample, filling an annulus between the core sample and the core sample holder with the oil and maintaining a confining pressure on the core sample in a range of from about 50 pounds per square inch (psi) to about 11,500 psi.

In some implementations, the method includes preparing the core sample prior to measuring the first electrical resistance of the core sample. Preparing the core sample can include: measuring a dry weight of the core sample; saturating the core sample with connate water; after saturating the core sample with connate water, measuring a wet weight of the core sample; and determining a pore volume of the core sample at least based on a density of the connate water and a difference between the wet weight and the dry weight of the core sample.

In some implementations, preparing the core sample includes: after determining the pore volume, centrifuging the core sample to drain at least a portion of the connate water from the core sample; after centrifuging the core sample, measuring a centrifuged weight of the core sample; and determining an initial water saturation of the core sample at least based on the density of the connate water and a difference between the wet weight and the centrifuged weight of the core sample.

In some implementations, measuring the first, second, third, and fourth electrical resistances of the core sample includes: applying an electric current across the core sample; and measuring an electrical response of the core sample in response to application of the electric current.

In some implementations, the electric current has a current in a range of from about −1 ampere to about 1 ampere.

In some implementations, the downhole reservoir temperature is in a range of from about 50 degrees Fahrenheit (° F.) to about 300° F., and the core sample is flushed with the oil while the core sample is heated to the downhole reservoir temperature until the pressure drop of the oil across the core sample has reached steady state.

In some implementations, the method includes, while flushing the core sample with the aqueous fluid, recording the amount of the oil displaced from the core sample, a pressure drop of the aqueous fluid across the core sample, and a flow rate of the aqueous fluid flushing the core sample as a function of time.

Certain aspects of the subject matter described can be implemented as a method. The method includes determining a pore volume of a core sample saturated with an aqueous fluid at least based on a density of the aqueous fluid, a wet weight of the core sample, and a dry weight of the core sample. The core sample is obtained from a subterranean formation containing hydrocarbons. The method includes draining at least a portion of the aqueous fluid from the core sample. The method includes determining an initial water saturation of the core sample at least based on the density of the aqueous fluid, the wet weight of the core sample, and a drained weight of the core sample. The method includes placing the core sample in a core sample holder. The method includes measuring a first electrical resistance of the core sample. The method includes flushing the core sample with oil to saturate the core sample with the oil. The method includes measuring a second electrical resistance of the core sample saturated with the oil. The method includes heating the core sample to a temperature that mimics a downhole temperature. The method includes pressurizing the oil in the core sample to a pressure that mimics a downhole pressure. The method includes measuring a third electrical resistance of the core sample. The method includes flushing the core sample with a second aqueous fluid to displace at least a portion of the oil from the core sample. The method includes, while flushing the core sample with the second aqueous fluid, measuring a fourth electrical resistance of the core sample and measuring an amount of the oil displaced from the core sample by the second aqueous fluid.

In some implementations, the method includes determining a change in pore throat of the core sample, permeability of the core sample, or both based on the measured first, second, third, and fourth electrical resistances of the core sample.

In some implementations, the method includes, after placing the core sample in the core sample holder and prior to measuring the first electrical resistance of the core sample, filling an annulus between the core sample and the core sample with the oil and maintaining a confining pressure on the core sample in a range of from about 50 pounds per square inch (psi) to about 11,500 psi.

In some implementations, measuring the first, second, third, and fourth electrical resistances of the core sample includes: applying an electric current across the core sample; and measuring an electrical response of the core sample in response to application of the electric current.

In some implementations, the electric current has a current in a range of from about −1 ampere to about 1 ampere.

In some implementations, the downhole reservoir temperature is in a range of from about 50 degrees Fahrenheit (° F.) to about 300° F., and the core sample is flushed with the oil while the core sample is heated to the downhole reservoir temperature until the pressure drop of the oil across the core sample has reached steady state.

In some implementations, the method includes, while flushing the core sample with the second aqueous fluid, recording the amount of the oil displaced from the core sample, a pressure drop of the aqueous fluid across the core sample, and a flow rate of the aqueous fluid flushing the core sample as a function of time.

Certain aspects of the subject matter described can be implemented as a system. The system includes an injection pump. The system includes an oil reservoir connected to the injection pump. The oil reservoir houses oil. The system includes a brine reservoir connected to the injection pump. The brine reservoir houses brine. The system includes a core sample holder connected to the oil reservoir and the brine reservoir. The core sample holder is configured to hold a core sample. The injection pump is configured to flow at least one of the oil from the oil reservoir or the brine from the brine reservoir through the core sample held by the core sample holder. The system includes a current-voltage analyzer connected to the core sample holder. The current-voltage analyzer is configured to apply an electric current across the core sample held by the core sample holder. The current-voltage analyzer is configured to measure an electrical response of the core sample held by the core sample holder in response to the current-voltage analyzer applying the electric current across the core sample held by the core sample holder. The system includes a confining pressure pump connected to the core sample holder. The confining pressure pump is configured to maintain a specified backpressure downstream of the core sample holder. The system includes a discharge container downstream of the core sample holder. The discharge container is positioned to receive at least a portion of fluid comprising the at least one of the oil or the brine that has flowed through the core sample held by the core sample.

In some implementations, the system includes: a computer; a pressure controller communicatively coupled to the computer and connected to the core sample holder; and a temperature controller communicatively coupled to the computer and connected to the core sample holder. The computer and the pressure controller can be cooperatively configured to adjust a desired operating pressure in the core sample holder. The computer and the temperature controller can be cooperatively configured to adjust a desired operating temperature in the core sample holder.

In some implementations, the computer is communicatively coupled to the current-voltage analyzer. The current-voltage analyzer can be configured to transmit a current signal to the computer that represents a current level of the electric current applied across the core sample. The current-voltage analyzer can be configured to transmit a response signal to the computer that represents the measured electrical response of the core sample.

The details of one or more implementations of the subject matter of this disclosure are set forth in the accompanying drawings and the description. Other features, aspects, and advantages of the subject matter will become apparent from the description, the drawings, and the claims.

### DESCRIPTION OF DRAWINGS

FIG. 1 is a schematic diagram of an example core flooding apparatus.

FIG. 2 is a flow chart of an example method for core flooding.

FIG. 3 is a flow chart of an example method for core flooding.

FIG. 4 is a block diagram of an example computer system.

### DETAILED DESCRIPTION

This disclosure describes a modified core flooding device which can be used to apply and measure electrical current. The core flooding device is configured to monitor and measure fluid flow simultaneously with the poromechanical response (behavior of fluid-saturated porous media). The core flooding device includes an injection pump, oil and brine reservoirs, a differential pressure meter, a core plug holder, an electric current analyzer, a backpressure pump, and a measuring cylinder. The core flooding device can be used to monitor change of pore throat size based on the extracted oil and pressure differential across the core plug. The behavior observed during experiments performed using the modified core flooding device can help to optimize the electrical current that will be implemented in electrically improved oil recovery (EIOR) in subterranean formations.

A common practice in the oil and gas industry is to inject water into a hydrocarbon reservoir to maintain its pressure and displace hydrocarbons to production wells. This injection of water is commonly referred to as secondary stage injection or secondary recovery. Seawater and aquifer water are some of the more widely used resources for injection. Injection of a second fluid in order to displace additional hydrocarbons after no more hydrocarbons are being extracted using the first fluid is referred to as tertiary stage injection or tertiary recovery. A remaining portion of the initial hydrocarbons in the reservoir can be extracted utilizing expensive enhanced recovery techniques, such as carbon dioxide (CO₂) injection or chemical flooding.

Regarding hydrocarbon production, the wettability of a reservoir can affect the hydrocarbon extraction process. Wettability is the tendency of a fluid to spread across or adhere to a solid surface in the presence of other immiscible fluids. In relation to the oil and gas industry, wettability can refer to the interaction between fluids such as hydrocarbons or water and a reservoir rock. A possible measure of wettability of a solid surface is defined by a contact angle of a fluid (such as a hydrocarbon droplet) with the surface (such as a rock formation) in the presence of another immiscible fluid (such as water or brine). Porous media, such as carbonate rock, can be complex and can have several configurations in different areas of the same formation due to the varied geometry and mineralogy of pore space. The wettability of such rock formation can therefore be heterogeneous, and heterogeneous wettability can further affect hydrocarbon recovery from rock formations in which hydrocarbons are trapped. A shift in wettability of a formation toward water-wetness can allow the extraction of additional hydrocarbons from the formation. Water flooding methods, can be considered to be a physical displacement method. The ionic composition of an aqueous brine solution, however, can trigger chemical interactions between a rock surface of a reservoir, oil, and the brine solution at a pore-scale level and can thereby alter wettability of the rock surface. In some cases, electrical stimulation can further promote a shift in wettability of the formation.

The subject matter described in this disclosure can be implemented in particular implementations, so as to realize one or more of the following advantages. Water contained in hydrocarbon reservoirs include various salts, and electrical stimulation of such reservoirs can change the behavior of fluids being extracted from hydrocarbon reservoirs. By electrically charging the core sample during core flooding experiments, pore throat size of the core sample can be adjusted and optimized to improve hydrocarbon extraction from the core sample during core flooding experiments. A pore throat of a rock is a pore space at the point where two grains meet, which connects two larger pore volumes. The number, size, and distribution of pore throats of a rock can control various characteristics of the rock, such as resistivity, flow, and capillary pressure. Increasing pore throat size can improve mobility of fluids through the rock, which can, in turn, improve hydrocarbon extraction from such rocks. The results from such core flooding experiments can be used to adjust and optimize parameters (such as electrical charging) out in the field to improve hydrocarbon extraction from hydrocarbon-containing reservoirs in subterranean formations. Electrical stimulation can also shift wettability of hydrocarbon-containing rock, which can further improve hydrocarbon extraction.

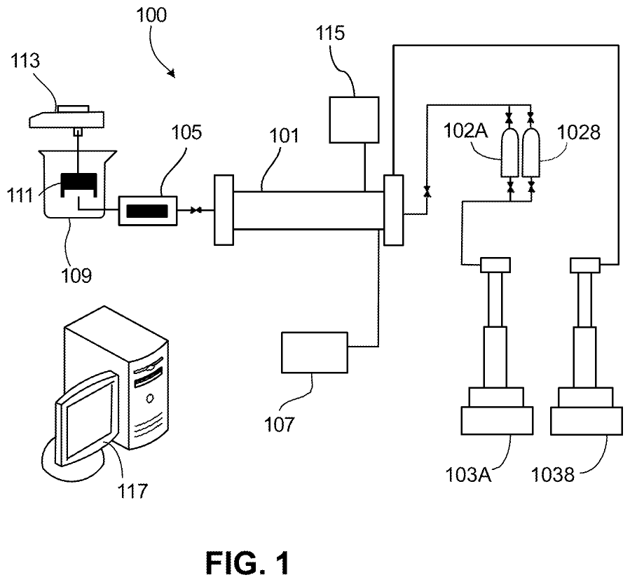

FIG. 1 illustrates an example of a core flooding apparatus 100. The core flooding apparatus 100 can include a core holder 101, an oil vessel 102A, a testing fluid vessel 102B, an injection pump 103A, a confining pressure pump 103B, a pressure controller 105, a temperature controller 107, a discharge container 109, an oil collector 111, a scale 113, a current-voltage analyzer 115, and a computer 117. A rock sample 120 can be secured in the core holder 101, and the core holder 101 can hold the rock sample 120 as a fluid is pumped through the rock sample 120. The rock sample 120 can be a rock sample obtained from a subterranean formation from which hydrocarbons are to be extracted. Analysis of the rock sample 120 can shed light on characteristics of a hydrocarbon reservoir of the subterranean formation. The rock sample 120 can have a cylindrical shape that can fit within the core holder 101. The oil vessel 102A can contain hydrocarbon fluid (for example, hydrocarbon oil from a rock formation). The testing fluid vessel 102B can contain a testing fluid (for example, connate water from a rock formation, seawater, or salinated treated wastewater).

The injection pump 103A can be used to pump fluid from the oil vessel 102A or the testing fluid vessel 102B and through the core holder 101 in order to flood the rock sample 120 with oil or testing fluid. By opening and closing various valves, the injection pump 103A pumps fluid from one vessel (102A or 102B), but not from both vessels (102A and 102B) simultaneously. The confining pressure pump 103B can be used to set a confining pressure against the rock sample 120 in the core holder 101 to mimic subsurface conditions. For example, the confining pressure can be within a range of approximately 50 pounds per square inch (psig) to 11,500 psi. The pressure controller 105 can regulate backpressure by controlling a valve, which can prevent fluid from reverse-flowing back into the rock sample 120. The pressure controller 105 and the confining pressure pump 103B can cooperate to adjust an operating pressure in the core holder 101. The operating pressure can be adjusted at various stages during a core flooding experiment. For example, for a portion of a core flooding experiment, the pressure controller 105 and the confining pressure pump 103B cooperate to maintain an operating pressure that mimics subsurface conditions (such as a downhole reservoir pressure). The temperature controller 107 can maintain an operating temperature that mimics subsurface conditions. For example, the temperature controller 107 can maintain a temperature within a range of approximately 10 degrees Celsius (° C.) to approximately 150° C. (approximately 50 degrees Fahrenheit (° F.) to approximately 300° F.). The temperature controller 107 can, for example, include an electric heater that converts electrical power into heat to adjust the operating temperature of the core flooding apparatus 100. The pressure controller 105, confining pressure pump 103B, and temperature controller 107 can work together to mimic subsurface conditions (for example, downhole conditions). Mimicking subsurface conditions can allow for the fluids in the rock sample 120 to behave more similarly as they would in the hydrocarbon-containing subterranean formation, such that the behavior and extractability of hydrocarbons from the subterranean formation can be more accurately estimated.

The discharge container 109 can hold the fluid exiting the core holder 101 through the backpressure valve. The oil collector 111 can be placed within the discharge container 109, such that the oil collector 111 can collect any oil that is discharged from the core holder 101. Since oil is less dense than water (and other similar aqueous solutions), the oil discharged from the core holder 101 can rise into the oil collector 111. The scale 113 can measure the weight of oil extracted from the rock sample after a core flooding test, and the scale 113 can be connected to the computer 117, which can use the weight (or change in weight) and known fluid densities of the oil and the testing fluid to calculate the amount of oil extracted from the rock sample 120.

The current-voltage analyzer 115 is connected to the core holder 101. The current-voltage analyzer 115 is communicatively coupled to the computer 117. The current-voltage analyzer 115 is configured to apply an electric current across the rock sample 120 held by the core holder 101. In some implementations, the electric current applied across the rock sample 120 by the current-voltage analyzer 115 is in a range of from about −1 ampere to about 1 ampere. In some implementations, the current-voltage analyzer 115 has a current resolution of about 10 microamperes—that is, the current-voltage analyzer 115 can generate and apply a current that can be adjusted in increments of about 10 microamperes. In some implementations, the current supplied by the current-voltage analyzer has a voltage of up to about 10 volts. The voltage sweeping applied by the current-voltage analyzer 115 can cause expansion of pore throat size of the rock sample 120. The rock sample 120 can exhibit an electrical response in response to the electric current applied by the current-voltage analyzer 115. The current-voltage analyzer 115 is configured to measure the electrical response of the rock sample 120. The current-voltage analyzer 115 is configured to transmit data (for example, the applied electric current and the electrical response of the rock sample 120) to the computer 117, which can then analyze the data. The core flooding apparatus 100 can be utilized to test a shift in wettability and/or a shift in permeability of the rock sample 120 associated with applying an electric current across the rock sample 120 during core flooding experiments. An increase in an Amott index value, an Amott-Harvey index value, or a USBM index value calculated from utilizing the core flooding apparatus 100 can signify a shift in wettability of the rock sample toward water-wetness. The core flooding apparatus 100 can also be utilized to test a change in oil recovery from the rock sample 120 associated with applying an electric current across the rock sample 120 during core flooding experiments. The core flooding apparatus 100 can be utilized to test various testing fluids in succession. For example, once steady-state is achieved (that is, no more significant oil extraction is observed) utilizing one testing fluid, another testing fluid can be used to flood the rock sample 120 in order to extract more oil from the rock sample 120.

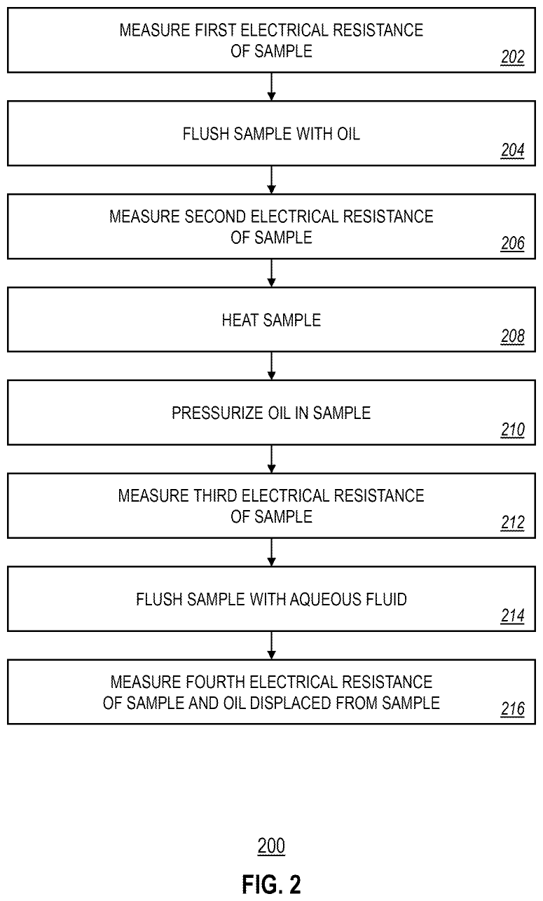

FIG. 2 is a flow chart of an example method 200 for performing a core flooding experiment on a core sample (such as the rock sample 120). The core flooding apparatus 100 can, for example, be used to implement the method 200. As described previously, the rock sample 120 is obtained from a subterranean formation that contains hydrocarbons. For example, the rock sample 120 is a core sample retrieved from a hydrocarbon reservoir. Prior to beginning the method 200, the pore volume of the core sample is at least partially filled with gas (for example, air). At block 202, a first electrical resistance of the rock sample 120 is measured. The first electrical resistance of the rock sample 120 can be measured at block 202, for example, by the current-voltage analyzer 115. Measuring the first electrical resistance of the rock sample 120 at block 202 can include applying an electric current across the rock sample 120 and measuring an electrical response of the rock sample 120 in response to application of the electric current. At block 204, the rock sample 120 is flushed with oil to displace the gas (for example, air) from the rock sample 120 and saturate the rock sample 120 with oil. The rock sample 120 can be flushed with oil at block 204, for example, by the injection pump 103A flowing oil from the oil vessel 102A to the rock sample 120. The rock sample 120 can be flushed with oil at block 204 until all of the gas that was in the pore volume of the rock sample 120 has been replaced with oil. The rock sample 120 can be flushed with oil at block 204 until a pressure drop of the oil across the rock sample 120 has reached steady state (for example, has stabilized and remains substantially constant). Stabilization of the pressure drop of the oil across the rock sample can signify that the rock sample 120 is saturated with oil, and the method 200 can proceed to block 206. At block 206, a second electrical resistance of the rock sample 120 saturated with the oil (block 204) is measured. The second electrical resistance of the rock sample 120 can be measured at block 206, for example, by the current-voltage analyzer 115. Measuring the second electrical resistance of the rock sample 120 at block 206 can include applying an electric current across the rock sample 120 and measuring an electrical response of the rock sample 120 in response to application of the electric current. At block 208, the rock sample 120 is heated to a downhole reservoir temperature. The rock sample 120 can be heated to the downhole reservoir temperature at block 210, for example, by the temperature controller 107. At block 210, the oil in the rock sample 120 is pressurized to a downhole reservoir pressure. The oil in the rock sample 120 can be pressurized to the downhole reservoir pressure at block 210, for example, by the pressure controller 105. At block 212, a third electrical resistance of the rock sample 120 is measured. The third electrical resistance of the rock sample 120 can be measured at block 212, for example, by the current-voltage analyzer 115. Measuring the second electrical resistance of the rock sample 120 at block 212 can include applying an electric current across the rock sample 120 and measuring an electrical response of the rock sample 120 in response to application of the electric current. At block 214, the rock sample 120 is flushed with an aqueous fluid (such as seawater or treated wastewater) to displace at least a portion of the oil from the rock sample 120. The rock sample 120 can be flushed with the aqueous fluid at block 214, for example, by the injection pump 103A flowing the aqueous fluid from the testing fluid vessel 102B to the rock sample 120. While the rock sample 120 is being flushed with the aqueous fluid at block 214, a fourth electrical resistance of the rock sample 120 is measured at block 216. The fourth electrical resistance of the rock sample 120 can be measured at block 216, for example, by the current-voltage analyzer 115. Measuring the second electrical resistance of the rock sample 120 at block 216 can include applying an electric current across the rock sample 120 and measuring an electrical response of the rock sample 120 in response to application of the electric current. While the rock sample 120 is being flushed with the aqueous fluid at block 214, an amount of the oil that is displaced from the rock sample 120 is measured at block 216. The amount of the oil that is displaced from the rock sample 120 can be measured at block 216, for example, by the scale 113 measuring the amount of oil that has accumulated in the oil collector 111. The rock sample 120 can be flushed with the aqueous fluid at block 214 until a pressure drop of the aqueous fluid across the rock sample 120 has reached steady state (for example, has stabilized and remains substantially constant). Block 216 can be repeated multiple times over the duration of block 214, for example, to analyze changes exhibited by the rock sample 120 over time in response to the electric current being applied across the rock sample 120. During voltage sweeping (e.g., blocks 202, 206, 212, 216), the electrical resistance of the rock sample 120 may drop when it reaches a threshold point for a pore throat. This threshold point can be measured and recorded for optimizing electrical stimulation of the rock sample 120 (and in turn, electrical stimulation of the subterranean formation).

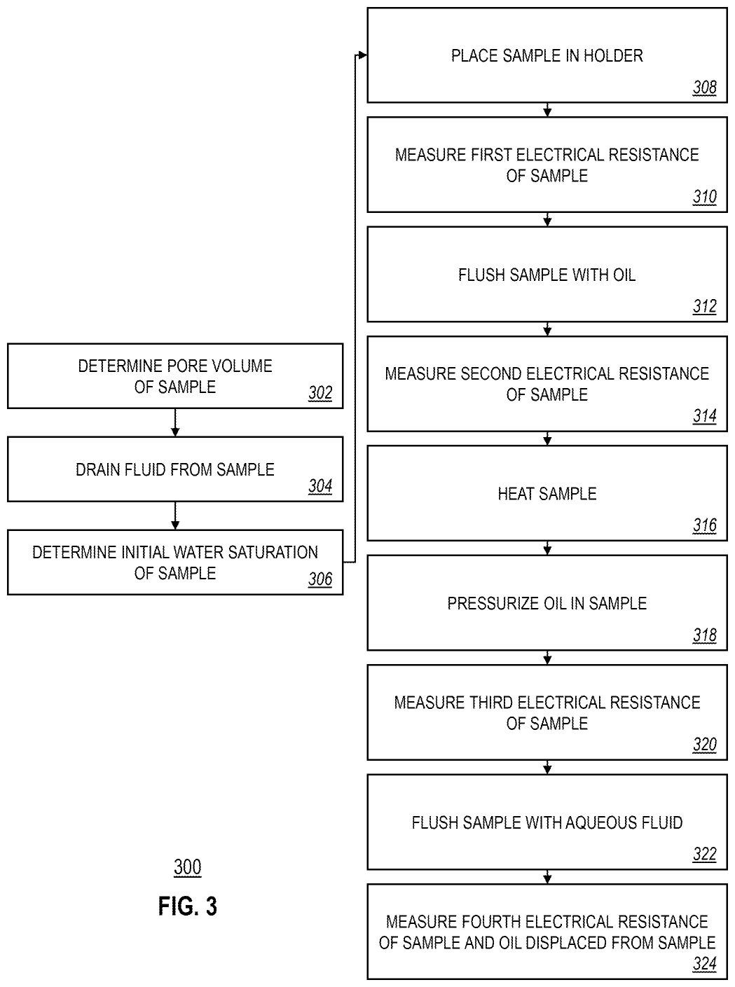

FIG. 3 is a flow chart of an example method 300 for performing a core flooding experiment on a core sample (such as the rock sample 120). The core flooding apparatus 100 can, for example, be used to implement the method 300. As described previously, the rock sample 120 is obtained from a subterranean formation that contains hydrocarbons. For example, the rock sample 120 is a core sample retrieved from a hydrocarbon reservoir. At block 302, a pore volume of the rock sample 120 is determined. Determining the pore volume of the rock sample 120 at block 302 can include measuring a dry weight of the rock sample 120, saturating the rock sample 120 with an aqueous fluid (such as connate water), and measuring a wet weight of the rock sample 120 that is saturated with the aqueous fluid. The pore volume of the rock sample 120 can be determined at block 302 by calculating the difference between the wet weight of the rock sample 120 and the dry weight of the rock sample 120 and then dividing the difference by the density of the aqueous fluid to obtain the pore volume. At block 304, at least a portion of the aqueous fluid is drained from the rock sample 120. For example, the rock sample 120 is centrifuged at block 304 to drain at least a portion of the aqueous fluid from the rock sample 120. At block 306, an initial water saturation of the rock sample 120 is determined. Determining the initial water saturation of the rock sample 120 at block 306 can include measuring a drained weight of the rock sample 120 (that is, the weight of the rock sample 120 after draining of the aqueous fluid from the rock sample 120). The initial water saturation of the rock sample 120 can be determined at block 306 by calculating the difference between the wet weight of the rock sample 120 and the drained weight of the rock sample 120 and then dividing the difference by the density of the aqueous fluid to obtain the initial water saturation. In some cases, the initial water saturation can be divided by the pore volume (determined at block 302) to obtain an initial water saturation percentage. Block 302, 304, and 306 are preparation steps that can be performed prior to each core flooding experiment.

The method 300 then proceeds to a core flooding portion of the method 300. The core flooding portion of the method 300 can be substantially similar to the method 200. At block 308, the rock sample 120 is placed in a core sample holder (such as the core holder 101). In some implementations, the rock sample 120 is wrapped in an insulating blanket (for example, a Teflon® blanket), and the wrapped core sample is surrounded with a polymer sleeve (such as a rubber sleeve) prior to being placed in the core holder 101 at block 308. At block 310, a first electrical resistance of the rock sample 120 is measured. The first electrical resistance of the rock sample 120 can be measured at block 310, for example, by the current-voltage analyzer 115. Measuring the first electrical resistance of the rock sample 120 at block 310 can include applying an electric current across the rock sample 120 and measuring an electrical response of the rock sample 120 in response to application of the electric current. At block 312, the rock sample 120 is flushed with oil to displace gas (for example, air) from the rock sample 120 and saturate the rock sample 120 with oil. The rock sample 120 can be flushed with oil at block 312, for example, by the injection pump 103A flowing oil from the oil vessel 102A to the rock sample 120. The rock sample 120 can be flushed with oil at block 312 until all of the gas that was in the pore volume of the rock sample 120 has been replaced with oil. The rock sample 120 can be flushed with oil at block 312 until a pressure drop of the oil across the rock sample 120 has reached steady state (for example, has stabilized and remains substantially constant). Stabilization of the pressure drop of the oil across the rock sample can signify that the rock sample 120 is saturated with oil, and the method 300 can proceed to block 314. At block 314, a second electrical resistance of the rock sample 120 saturated with oil (block 312) is measured. The second electrical resistance of the rock sample 120 can be measured at block 314, for example, by the current-voltage analyzer 115. Measuring the second electrical resistance of the rock sample 120 at block 314 can include applying an electric current across the rock sample 120 and measuring an electrical response of the rock sample 120 in response to application of the electric current. At block 316, the rock sample 120 is heated to a downhole reservoir temperature. The rock sample 120 can be heated to the downhole reservoir temperature at block 316, for example, by the temperature controller 107. At block 318, the oil in the rock sample 120 is pressurized to a downhole reservoir pressure. The oil in the rock sample 120 can be pressurized to the downhole reservoir pressure at block 318, for example, by the pressure controller 105. At block 320, a third electrical resistance of the rock sample 120 is measured. The third electrical resistance of the rock sample 120 can be measured at block 320, for example, by the current-voltage analyzer 115. Measuring the third electrical resistance of the rock sample 120 at block 320 can include applying an electric current across the rock sample 120 and measuring an electrical response of the rock sample 120 in response to application of the electric current. At block 322, the rock sample 120 is flushed with an aqueous fluid (such as seawater or treated wastewater) to displace at least a portion of the oil from the rock sample 120. The rock sample 120 can be flushed with the aqueous fluid at block 322, for example, by the injection pump 103A flowing the aqueous fluid from the testing fluid vessel 102B to the rock sample 120. While the rock sample 120 is being flushed with the aqueous fluid at block 322, a fourth electrical resistance of the rock sample 120 is measured at block 324. The fourth electrical resistance of the rock sample 120 can be measured at block 324, for example, by the current-voltage analyzer 115. Measuring the fourth electrical resistance of the rock sample 120 at block 324 can include applying an electric current across the rock sample 120 and measuring an electrical response of the rock sample 120 in response to application of the electric current. While the rock sample 120 is being flushed with the aqueous fluid at block 322, an amount of the oil that is displaced from the rock sample 120 is measured at block 324. The amount of the oil that is displaced from the rock sample 120 can be measured at block 324, for example, by the scale 113 measuring the amount of oil that has accumulated in the oil collector 111. The rock sample 120 can be flushed with the aqueous fluid at block 322 until a pressure drop of the aqueous fluid across the rock sample 120 has reached steady state (for example, has stabilized and remains substantially constant). Block 324 can be repeated multiple times over the duration of block 322, for example, to analyze changes exhibited by the rock sample 120 over time in response to the electric current being applied across the rock sample 120. During voltage sweeping (e.g., blocks 310, 314, 320, 324), the electrical resistance of the rock sample 120 may drop when it reaches a threshold point for a pore throat. This threshold point can be measured and recorded for optimizing electrical stimulation of the rock sample 120 (and in turn, electrical stimulation of the subterranean formation).

The methods 200 and 300 can be implemented to optimize operating parameters for extracting hydrocarbons from a well formed in a subterranean formation. As mentioned previously, the electrical resistance of the rock sample 120 can be measured and recorded when the rock sample 120 reaches the threshold (breaking) point for the rock sample's pore throat. Based on the known electrode contact area of the current-voltage analyzer 115 and known length of the rock sample 120, electrical resistivity (p) of the rock sample 120 can be calculated by Equation 1:

$$R = \frac{V}{I} = \frac{ρ \times L}{A} \qquad ( 1 )$$

where R is resistance, V is voltage, I is current, L is length of the rock sample 120, and A is the electrode contact area. The electrical stimulation of the well can be adjusted based on these calculations for improving hydrocarbon extraction from the well.

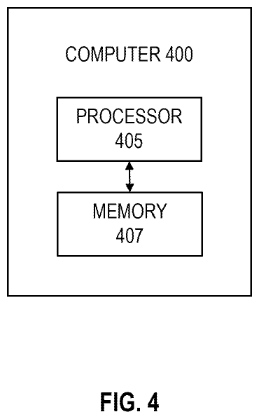

FIG. 4 is a block diagram of an example computer 400 used to provide computational functionalities associated with described algorithms, methods, functions, processes, flows, and procedures, as described in this specification, according to an implementation. The computer 117 (shown in FIG. 1) can, for example, be an implementation of the computer 400. The illustrated computer 400 is intended to encompass any computing device such as a server, desktop computer, laptop/notebook computer, one or more processors within these devices, or any other processing device, including physical or virtual instances (or both) of the computing device. Additionally, the computer 400 can include a computer that includes an input device, such as a keypad, keyboard, touch screen, or other device that can accept user information, and an output device that conveys information associated with the operation of the computer 400, including digital data, visual, audio information, or a combination of information.

The computer 400 includes a processor 405. The processor 405 may be a microprocessor, a multi-core processor, a multithreaded processor, an ultra-low-voltage processor, an embedded processor, or a virtual processor. In some embodiments, the processor 405 may be part of a system-on-a-chip (SoC) in which the processor 405 and the other components of the computer 400 are formed into a single integrated electronics package. In some implementations, the processor 405 may include processors from Intel® Corporation of Santa Clara, California, from Advanced Micro Devices, Inc. (AMD) of Sunnyvale, California, or from ARM Holdings, LTD., Of Cambridge, England. Any number of other processors from other suppliers may also be used. Although illustrated as a single processor 405 in FIG. 4, two or more processors may be used according to particular needs, desires, or particular implementations of the computer 400. Generally, the processor 405 executes instructions and manipulates data to perform the operations of the computer 400 and any algorithms, methods, functions, processes, flows, and procedures as described in this specification.

The computer 400 also includes a memory 407 that can hold data for the computer 400 or other components (or a combination of both) that can be connected to the network. Although illustrated as a single memory 407 in FIG. 4, two or more memories 407 (of the same or combination of types) can be used according to particular needs, desires, or particular implementations of the computer 400 and the described functionality. While memory 407 is illustrated as an integral component of the computer 400, memory 407 can be external to the computer 400. The memory 407 can be a transitory or non-transitory storage medium. In some implementations, such as in PLCs and other process control units, the memory 407 is integrated with the database 406 used for long-term storage of programs and data. The memory 407 can include any number of volatile and nonvolatile memory devices, such as volatile random-access memory (RAM), static random-access memory (SRAM), flash memory, and the like. In smaller devices, such as PLCs, the memory 407 may include registers associated with the processor 405 itself.

The memory 407 stores computer-readable instructions executable by the processor 405 that, when executed, cause the processor 405 to perform operations, such as transmitting a signal to the injection pump 103A to adjust a flow rate of fluid being flowed to the rock sample 120, transmitting a signal to the pressure controller 105 to adjust the backpressure for the core flooding apparatus 100, transmitting a signal to the temperature controller 107 to adjust the operating temperature of the core flooding apparatus 100, and transmitting a signal to the current-voltage analyzer 107 to adjust the current supplied by the current-voltage analyzer 107 across the rock sample 120. The computer 400 can also include a power supply 414. The power supply 414 can include a rechargeable or non-rechargeable battery that can be configured to be either user- or non-user-replaceable. The power supply 414 can be hard-wired. There may be any number of computers 400 associated with, or external to, a computer system containing computer 400, each computer 400 communicating over the network. Further, the term “client,” “user,” “operator,” and other appropriate terminology may be used interchangeably, as appropriate, without departing from this specification. Moreover, this specification contemplates that many users may use one computer 400, or that one user may use multiple computers 400.

The computational operations can be implemented using one or more databases, which store data received from the physical world operations and/or generated internally within the computational operations (e.g., by implementing the methods of the present disclosure) or both. For example, the one or more computers 400 process inputs from the physical world operations to assess conditions in the physical world, the outputs of which are stored in the databases. The source and received signals are provided to the computational operations where they are stored in the databases and analyzed by the one or more computers 400.

In some implementations, one or more outputs generated by the one or more computers 400 can be provided as feedback/input to the physical world operations (either as direct input or stored in the databases). The physical world operations can use the feedback/input to control physical components used to perform the physical world operations in the real world, such as adjusting the percent opening of a valve or adjusting a speed of a pump.

In some implementations of the computational operations, customized user interfaces can present intermediate or final results of the above-described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or app), or at a central processing facility. The presented information can include feedback, such as changes in parameters or processing inputs, that the user can select to improve an operation environment, such as in the testing or operation of petrochemical processes or facilities. For example, the feedback can include parameters that, when selected by the user, can cause a change to, or an improvement in, operating parameters (such as flow control and/or operating conditions (such as pressure and temperature) in the system 100). The feedback, when implemented by the user, can improve the speed and accuracy of calculations, streamline processes, improve models, and solve problems related to efficiency, performance, safety, reliability, costs, downtime, and the need for human interaction.

In some implementations, the feedback can be implemented in real-time, such as to provide an immediate or near-immediate change in operations or in a model. The term real-time (or similar terms as understood by one of ordinary skill in the art) means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second (s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

Events can include readings or measurements captured by equipment such as sensors, pumps, heaters, or other equipment. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas processing. For example, outputs of the present disclosure can be used as inputs to other equipment and/or systems at a facility. This can be especially useful for systems or various pieces of equipment that are located several meters or several miles apart, or are located in different countries or other jurisdictions.

### Example

For an example core flooding experiment, a core sample was prepared as followed. The dry weight of the core sample was measured. The core sample was then saturated with connate water under a vacuum for 5 to 7 days to achieve ionic equilibrium with the core sample. The saturated core sample was then weighed to obtain a wet weight of the core sample. The pore volume of the core sample was calculated by obtaining the weight difference (wet weight minus dry weight) and dividing the weight difference by the density of the connate water (at room temperature). The core sample was then centrifuged at a rotation rate at 5,000 revolutions per minute for 12 hours to drain the connate water from the core sample and establish the initial water saturation. After centrifuging, the core sample was weighed to obtain a centrifuged weight of the core sample. The initial water saturation of the core sample was estimated by obtaining the weight difference (wet weight minus centrifuged weight) and dividing the weight difference by the density of the connate water (at room temperature).

Prior to beginning the example core flooding experiment, the oil vessel and the testing fluid vessel were filled with dead oil and brine, respectively. The discharge container and oil container were checked, and the scale was calibrated to ensure accurate measurement of oil production from the core sample during the core flooding experiment. The core sample was wrapped in a Teflon® insulating blanket, placed into a rubber sleeve, and loaded into the core holder. The annulus of the core holder surrounding the core sample was filled with dead oil and a confining pressure in a range of from 50 to 11,500 psi was maintained. The pressure controller was initialized to regulate backpressure on the core sample in a range of from 0 to 5,000 psi. Once set up, an initial electrical resistance of the core sample was measured. Dead oil was then flushed through the core sample at backpressure conditions to displace gas (air) and ensure complete oil saturation. Dead oil flushing was maintained for 1 to 2 weeks until the pressure drop across the core sample stabilized. After dead oil flushing, the electrical resistance of the core sample was measured again. The temperature controller then maintained an operating temperature in a range of from 50° F. to 300° F. The core sample was aged at this operating temperature with repeated dead oil flushing for 1 to 2 weeks until the pressure drop across the core sample stabilized. The pore pressure of the core sample was set to reservoir pressure by the pressure controller regulating backpressure. The electrical resistance of the core sample was measured again. Seawater flooding was then conducted. During seawater flooding, the amount of oil produced from the core sample, the pressure drop across the core sample, and the injection rate of the seawater was measured and recorded as a function of time. The amount of produced oil, pressure drop, seawater injection rate, or any combinations of these can be plotted on a graph as a function of time to analyze the behavior of fluid flowing through and/or out of the core sample. The electrical resistance of the core sample was also measured and recorded as a function of time during seawater flooding. The injection rate of the seawater was increased to 2 cubic centimeters per minute (cc/min) and then to 4 cc/min to ensure that all mobile oil from the core sample was extracted. The oil production from the core sample, water production from the core sample, and electrical resistance of the core sample were measured and recorded as a function of time during the fluid injection.

### Embodiments

In an example implementation (or aspect), a method comprises: measuring a first electrical resistance of a core sample placed in a core sample holder, wherein the core sample is obtained from a subterranean formation containing hydrocarbons, wherein the core sample is in a dry state, wherein a pore volume of the core sample is at least partially filled with gas; flushing the core sample with oil to displace gas from the core sample and saturate the core sample with the oil; after saturating the core sample with the oil, measuring a second electrical resistance of the core sample saturated with the oil; heating the core sample to a downhole reservoir temperature; pressurizing the oil in the core sample to a downhole reservoir pressure; after pressurizing the oil, measuring a third electrical resistance of the core sample; flushing the core sample with an aqueous fluid to displace at least a portion of the oil from the core sample; and while flushing the core sample with the aqueous fluid, measuring a fourth electrical resistance of the core sample and measuring an amount of the oil displaced from the core sample.

In an example implementation (or aspect) combinable with any other example implementation (or aspect), the method further comprises determining a change in pore throat of the core sample, permeability of the core sample, or both based on the measured first, second, third, and fourth electrical resistances of the core sample.

In an example implementation (or aspect) combinable with any other example implementation (or aspect), the method further comprises, prior to measuring the first electrical resistance of the core sample: wrapping the core sample in an insulating blanket; surrounding the wrapped core sample with a polymer sleeve; and placing the wrapped core sample surrounded by the polymer sleeve in the core sample holder.

In an example implementation (or aspect) combinable with any other example implementation (or aspect), the method further comprises, prior to measuring the first electrical resistance of the core sample, filling an annulus between the core sample and the core sample holder with the oil and maintaining a confining pressure on the core sample in a range of from about 50 pounds per square inch (psi) to about 11,500 psi.

In an example implementation (or aspect) combinable with any other example implementation (or aspect), the method further comprises preparing the core sample prior to measuring the first electrical resistance of the core sample, wherein preparing the core sample comprises: measuring a dry weight of the core sample; saturating the core sample with connate water; after saturating the core sample with connate water, measuring a wet weight of the core sample; and determining a pore volume of the core sample at least based on a density of the connate water and a difference between the wet weight and the dry weight of the core sample.

In an example implementation (or aspect) combinable with any other example implementation (or aspect), preparing the core sample further comprises: after determining the pore volume, centrifuging the core sample to drain at least a portion of the connate water from the core sample; after centrifuging the core sample, measuring a centrifuged weight of the core sample; and determining an initial water saturation of the core sample at least based on the density of the connate water and a difference between the wet weight and the centrifuged weight of the core sample.

In an example implementation (or aspect) combinable with any other example implementation (or aspect), measuring the first, second, third, and fourth electrical resistances of the core sample comprises: applying an electric current across the core sample; and measuring an electrical response of the core sample in response to application of the electric current.

In an example implementation (or aspect) combinable with any other example implementation (or aspect), the electric current has a current in a range of from about-1 ampere to about 1 ampere.

In an example implementation (or aspect) combinable with any other example implementation (or aspect), the downhole reservoir temperature is in a range of from about 50 degrees Fahrenheit (° F.) to about 300° F., and the core sample is flushed with the oil while the core sample is heated to the downhole reservoir temperature until the pressure drop of the oil across the core sample has reached steady state.

In an example implementation (or aspect) combinable with any other example implementation (or aspect), the method further comprises, while flushing the core sample with the aqueous fluid, recording the amount of the oil displaced from the core sample, a pressure drop of the aqueous fluid across the core sample, and a flow rate of the aqueous fluid flushing the core sample as a function of time.

In an example implementation (or aspect), a method comprises: determining a pore volume of a core sample saturated with an aqueous fluid at least based on a density of the aqueous fluid, a wet weight of the core sample, and a dry weight of the core sample, wherein the core sample is obtained from a subterranean formation containing hydrocarbons; draining at least a portion of the aqueous fluid from the core sample; determining an initial water saturation of the core sample at least based on the density of the aqueous fluid, the wet weight of the core sample, and a drained weight of the core sample; placing the core sample in a core sample holder; measuring a first electrical resistance of the core sample; flushing the core sample with oil to saturate the core sample with the oil; measuring a second electrical resistance of the core sample saturated with the oil; heating the core sample to a temperature that mimics a downhole temperature; pressurizing the oil in the core sample to a pressure that mimics a downhole pressure; measuring a third electrical resistance of the core sample; flushing the core sample with a second aqueous fluid to displace at least a portion of the oil from the core sample; and while flushing the core sample with the second aqueous fluid, measuring a fourth electrical resistance of the core sample and measuring an amount of the oil displaced from the core sample by the second aqueous fluid.

In an example implementation (or aspect) combinable with any other example implementation (or aspect), the method further comprises determining a change in pore throat of the core sample, permeability of the core sample, or both based on the measured first, second, third, and fourth electrical resistances of the core sample.

In an example implementation (or aspect) combinable with any other example implementation (or aspect), the method further comprises, after placing the core sample in the core sample holder and prior to measuring the first electrical resistance of the core sample, filling an annulus between the core sample and the core sample with the oil and maintaining a confining pressure on the core sample in a range of from about 50 pounds per square inch (psi) to about 11,500 psi.

In an example implementation (or aspect) combinable with any other example implementation (or aspect), measuring the first, second, third, and fourth electrical resistances of the core sample comprises: applying an electric current across the core sample; and measuring an electrical response of the core sample in response to application of the electric current.

In an example implementation (or aspect) combinable with any other example implementation (or aspect), the electric current has a current in a range of from about-1 ampere to about 1 ampere.

In an example implementation (or aspect) combinable with any other example implementation (or aspect), the downhole reservoir temperature is in a range of from about 50 degrees Fahrenheit (° F.) to about 300° F., and the core sample is flushed with the oil while the core sample is heated to the downhole reservoir temperature until the pressure drop of the oil across the core sample has reached steady state.

In an example implementation (or aspect) combinable with any other example implementation (or aspect), the method further comprises, while flushing the core sample with the second aqueous fluid, recording the amount of the oil displaced from the core sample, a pressure drop of the aqueous fluid across the core sample, and a flow rate of the aqueous fluid flushing the core sample as a function of time.

In an example implementation (or aspect), a system comprises: an injection pump; an oil reservoir connected to the injection pump, the oil reservoir housing oil; a brine reservoir connected to the injection pump, the brine reservoir housing brine; a core sample holder connected to the oil reservoir and the brine reservoir, the core sample holder configured to hold a core sample, the injection pump is configured to flow at least one of the oil from the oil reservoir or the brine from the brine reservoir through the core sample held by the core sample holder; a current-voltage analyzer connected to the core sample holder, the current-voltage analyzer configured to apply an electric current across the core sample held by the core sample holder, the current-voltage analyzer configured to measure an electrical response of the core sample held by the core sample holder in response to the current-voltage analyzer applying the electric current across the core sample held by the core sample holder; a confining pressure pump connected to the core sample holder, the confining pressure pump configured to maintain a specified backpressure downstream of the core sample holder; and a discharge container downstream of the core sample holder, the discharge container positioned to receive at least a portion of fluid comprising the at least one of the oil or the brine that has flowed through the core sample held by the core sample.

In an example implementation (or aspect) combinable with any other example implementation (or aspect), the system further comprises: a computer; a pressure controller communicatively coupled to the computer and connected to the core sample holder, wherein the computer and the pressure controller are cooperatively configured to adjust a desired operating pressure in the core sample holder; and a temperature controller communicatively coupled to the computer and connected to the core sample holder, wherein the computer and the temperature controller are cooperatively configured to adjust a desired operating temperature in the core sample holder.

In an example implementation (or aspect) combinable with any other example implementation (or aspect), the computer is communicatively coupled to the current-voltage analyzer, wherein the current-voltage analyzer is configured to transmit a current signal to the computer that represents a current level of the electric current applied across the core sample, wherein the current-voltage analyzer is configured to transmit a response signal to the computer that represents the measured electrical response of the core sample.

While this specification contains many specific implementation details, these should not be construed as limitations on the scope of what may be claimed, but rather as descriptions of features that may be specific to particular implementations. Certain features that are described in this specification in the context of separate implementations can also be implemented, in combination, in a single implementation. Conversely, various features that are described in the context of a single implementation can also be implemented in multiple implementations, separately, or in any sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

As used in this disclosure, the terms “a,” “an,” or “the” are used to include one or more than one unless the context clearly dictates otherwise. The term “or” is used to refer to a nonexclusive “or” unless otherwise indicated. The statement “at least one of A and B” has the same meaning as “A, B, or A and B.” In addition, it is to be understood that the phraseology or terminology employed in this disclosure, and not otherwise defined, is for the purpose of description only and not of limitation. Any use of section headings is intended to aid reading of the document and is not to be interpreted as limiting; information that is relevant to a section heading may occur within or outside of that particular section.

As used in this disclosure, the term “about” or “approximately” can allow for a degree of variability in a value or range, for example, within 10%, within 5%, or within 1% of a stated value or of a stated limit of a range.

As used in this disclosure, the term “substantially” refers to a majority of, or mostly, as in at least about 50%, 60%, 70%, 80%, 90%, 95%, 96%, 97%, 98%, 99%, 99.5%, 99.9%, 99.99%, or at least about 99.999% or more.

Values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “0.1% to about 5%” or “0.1% to 5%” should be interpreted to include about 0.1% to about 5%, as well as the individual values (for example, 1%, 2%, 3%, and 4%) and the sub-ranges (for example, 0.1% to 0.5%, 1.1% to 2.2%, 3.3% to 4.4%) within the indicated range. The statement “X to Y” has the same meaning as “about X to about Y,” unless indicated otherwise. Likewise, the statement “X, Y, or Z” has the same meaning as “about X, about Y, or about Z,” unless indicated otherwise.

Particular implementations of the subject matter have been described. Other implementations, alterations, and permutations of the described implementations are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results. In certain circumstances, multitasking or parallel processing (or a combination of multitasking and parallel processing) may be advantageous and performed as deemed appropriate.

Moreover, the separation or integration of various system modules and components in the previously described implementations should not be understood as requiring such separation or integration in all implementations, and it should be understood that the described components and systems can generally be integrated together or packaged into multiple products.

Accordingly, the previously described example implementations do not define or constrain the present disclosure. Other changes, substitutions, and alterations are also possible without departing from the spirit and scope of the present disclosure.

What is claimed is:

1. A method comprising:
measuring a first electrical resistance of a core sample placed in a core sample holder, wherein the core sample is obtained from a subterranean formation containing hydrocarbons, wherein the core sample is in a dry state, wherein a pore volume of the core sample is at least partially filled with gas;
flushing the core sample with oil to displace gas from the core sample and saturate the core sample with the oil;
after saturating the core sample with the oil, measuring a second electrical resistance of the core sample saturated with the oil;
heating the core sample to a downhole reservoir temperature;
pressurizing the oil in the core sample to a downhole reservoir pressure;
after pressurizing the oil, measuring a third electrical resistance of the core sample;
flushing the core sample with an aqueous fluid to displace at least a portion of the oil from the core sample; and
while flushing the core sample with the aqueous fluid, measuring a fourth electrical resistance of the core sample and measuring an amount of the oil displaced from the core sample.

2. The method of claim 1, further comprising determining a change in pore throat of the core sample, permeability of the core sample, or both based on the measured first, second, third, and fourth electrical resistances of the core sample.

3. The method of claim 2, further comprising, prior to measuring the first electrical resistance of the core sample:
wrapping the core sample in an insulating blanket;
surrounding the wrapped core sample with a polymer sleeve; and
placing the wrapped core sample surrounded by the polymer sleeve in the core sample holder.

4. The method of claim 3, further comprising, prior to measuring the first electrical resistance of the core sample, filling an annulus between the core sample and the core sample holder with the oil and maintaining a confining pressure on the core sample in a range of from about 50 pounds per square inch (psi) to about 11,500 psi.

5. The method of claim 4, further comprising preparing the core sample prior to measuring the first electrical resistance of the core sample, wherein preparing the core sample comprises:
measuring a dry weight of the core sample;
saturating the core sample with connate water;
after saturating the core sample with connate water, measuring a wet weight of the core sample; and
determining a pore volume of the core sample at least based on a density of the connate water and a difference between the wet weight and the dry weight of the core sample.

6. The method of claim 5, wherein preparing the core sample further comprises:
after determining the pore volume, centrifuging the core sample to drain at least a portion of the connate water from the core sample;
after centrifuging the core sample, measuring a centrifuged weight of the core sample; and
determining an initial water saturation of the core sample at least based on the density of the connate water and a difference between the wet weight and the centrifuged weight of the core sample.

7. The method of claim 6, wherein measuring the first, second, third, and fourth electrical resistances of the core sample comprises:
applying an electric current across the core sample; and
measuring an electrical response of the core sample in response to application of the electric current.

8. The method of claim 7, wherein the electric current has a current in a range of from about-1 ampere to about 1 ampere.

9. The method of claim 8, wherein the downhole reservoir temperature is in a range of from about 50 degrees Fahrenheit (° F.) to about 300° F., and the core sample is flushed with the oil while the core sample is heated to the downhole reservoir temperature until the pressure drop of the oil across the core sample has reached steady state.

10. The method of claim 9, further comprising, while flushing the core sample with the aqueous fluid, recording the amount of the oil displaced from the core sample, a pressure drop of the aqueous fluid across the core sample, and a flow rate of the aqueous fluid flushing the core sample as a function of time.

11. A method comprising:
determining a pore volume of a core sample saturated with an aqueous fluid at least based on a density of the aqueous fluid, a wet weight of the core sample, and a dry weight of the core sample, wherein the core sample is obtained from a subterranean formation containing hydrocarbons;
draining at least a portion of the aqueous fluid from the core sample;
determining an initial water saturation of the core sample at least based on the density of the aqueous fluid, the wet weight of the core sample, and a drained weight of the core sample;
placing the core sample in a core sample holder;
measuring a first electrical resistance of the core sample;
flushing the core sample with oil to saturate the core sample with the oil;
measuring a second electrical resistance of the core sample saturated with the oil;
heating the core sample to a temperature that mimics a downhole temperature;
pressurizing the oil in the core sample to a pressure that mimics a downhole pressure;
measuring a third electrical resistance of the core sample;
flushing the core sample with a second aqueous fluid to displace at least a portion of the oil from the core sample; and
while flushing the core sample with the second aqueous fluid, measuring a fourth electrical resistance of the core sample and measuring an amount of the oil displaced from the core sample by the second aqueous fluid.

12. The method of claim 11, further comprising determining a change in pore throat of the core sample, permeability of the core sample, or both based on the measured first, second, third, and fourth electrical resistances of the core sample.

13. The method of claim 12, further comprising, after placing the core sample in the core sample holder and prior to measuring the first electrical resistance of the core sample, filling an annulus between the core sample and the core sample with the oil and maintaining a confining pressure on the core sample in a range of from about 50 pounds per square inch (psi) to about 11,500 psi.

14. The method of claim 13, wherein measuring the first, second, third, and fourth electrical resistances of the core sample comprises:
applying an electric current across the core sample; and
measuring an electrical response of the core sample in response to application of the electric current.

15. The method of claim 14, wherein the electric current has a current in a range of from about-1 ampere to about 1 ampere.

16. The method of claim 15, wherein the downhole reservoir temperature is in a range of from about 50 degrees Fahrenheit (° F.) to about 300° F., and the core sample is flushed with the oil while the core sample is heated to the downhole reservoir temperature until the pressure drop of the oil across the core sample has reached steady state.

17. The method of claim 16, further comprising, while flushing the core sample with the second aqueous fluid, recording the amount of the oil displaced from the core sample, a pressure drop of the aqueous fluid across the core sample, and a flow rate of the aqueous fluid flushing the core sample as a function of time.

18. A system comprising:
an injection pump;
an oil reservoir connected to the injection pump, the oil reservoir housing oil;
a brine reservoir connected to the injection pump, the brine reservoir housing brine;
a core sample holder connected to the oil reservoir and the brine reservoir, the core sample holder configured to hold a core sample, the injection pump is configured to flow at least one of the oil from the oil reservoir or the brine from the brine reservoir through the core sample held by the core sample holder;
a current-voltage analyzer connected to the core sample holder, the current-voltage analyzer configured to apply an electric current across the core sample held by the core sample holder, the current-voltage analyzer configured to measure an electrical response of the core sample held by the core sample holder in response to the current-voltage analyzer applying the electric current across the core sample held by the core sample holder;
a confining pressure pump connected to the core sample holder, the confining pressure pump configured to maintain a specified backpressure downstream of the core sample holder; and
a discharge container downstream of the core sample holder, the discharge container positioned to receive at least a portion of fluid comprising the at least one of the oil or the brine that has flowed through the core sample held by the core sample.

19. The system of claim 18, further comprising:
a computer;
a pressure controller communicatively coupled to the computer and connected to the core sample holder, wherein the computer and the pressure controller are cooperatively configured to adjust a desired operating pressure in the core sample holder; and
a temperature controller communicatively coupled to the computer and connected to the core sample holder, wherein the computer and the temperature controller are cooperatively configured to adjust a desired operating temperature in the core sample holder.

20. The system of claim 19, wherein the computer is communicatively coupled to the current-voltage analyzer, wherein the current-voltage analyzer is configured to transmit a current signal to the computer that represents a current level of the electric current applied across the core sample, wherein the current-voltage analyzer is configured to transmit a response signal to the computer that represents the measured electrical response of the core sample.
